(19) United States
(12) Patent Application Publication
(10) Pub. No.: US 2026/0315565 A1
(43) Pub. Date: Oct. 15, 2026

(54) ANTIOXIDANT-CONTAINING EYE DROP FORMULATIONS AND METHODS OF PREVENTION AND/OR TREATMENT USING SAME

(71) Applicant: Marizyme, Inc., Jupiter, FL (US)

(72) Inventor: Catherine Pachuk, Jupiter, FL (US)

(21) Appl. No.: 19/106,879

(22) PCT Filed: Aug. 25, 2023
(86) PCT No.: PCT/US2023/031113

§ 371 Date: Feb. 26, 2025

Related U.S. Application Data
(60) Provisional application No. 63/401,486, filed on Aug. 26, 2022.

(51) Int. Cl. A61K 38/06; A61K 9/08; A61K 31/01; A61K 31/015; A61K 31/047; A61K 31/355; A61K 31/375; A61K 33/04; A61K 33/30; A61K 38/05; A61K 47/02

(52) U.S. Cl. CPC A61K 38/063; A61K 9/08; A61K 31/01; A61K 31/015; A61K 31/047; A61K 31/355; A61K 31/375; A61K 33/04; A61K 33/30; A61K 38/05; A61K 47/02

(57) ABSTRACT

An aqueous antioxidant-containing formulation, including: at least one antioxidant selected from L-ascorbic acid, L-glutathione, cysteinylglycine, vitamin C, vitamin E, zinc, selenium, beta-carotene, lycopene, lutein, and zeaxanthin; and a physiologically acceptable salt solution. A method of preventing and/or treating ocular oxidative damage and/or stress by administration of the aqueous antioxidant-containing formulation to a patient in need thereof.

### TECHNICAL FIELD

Antioxidant-containing eye drop formulations to protect eyes against oxidative damage and/or stress, maintain and support general eye health, and methods of preventing and/or treating oxidative damage and/or stress in the eye using the antioxidant-containing eye drop formulations. The eye drops are also ionically balanced and isotonic.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions, or is known to be relevant to an attempt to solve any problem with which this specification is concerned.

Eyes are very susceptible to oxidative damage and/or stress. Oxidative damage and/or stress is involved in the pathogenesis of many eye diseases, especially those that occur in the aging population. Examples of eye diseases arising from oxidative damage and/or stress include cataract, macular degeneration, uveitis, retinopathy, keratitis, ocular inflammation, dry eye disease, glaucoma, eye infection, and the like, but are not limited thereto.

The global eye health supplements market size was valued at USD 1.5 billion in 2019 and is expected to expand at a compound annual growth rate (CAGR) of 6.1% from 2020 to 2027. The growing incidences of vision impairment and rising myopic population, especially among the aging population, is driving the market. High myopia can be attributable to increased risk of various progressive eye disorders such as cataract, macular degeneration, uveitis, retinopathy, keratitis, ocular inflammation, dry eye disease, glaucoma, and the like, all of which are considered diseases of oxidative damage. Increasing preference for eye health supplements among the older group of individuals and millennials highly prone to digital eye fatigue and myopia is further driving the market worldwide. Increase in prevalence of various eye conditions including Age-related Macular Degeneration (AMD), dry eye syndrome, cataract, and inflammation along with the growing recommendation for eye health supplements by ophthalmologists for prevention and treatment of these disorders is also a contributing factor for the growth of this market.

Currently there are no antioxidant eye drop products to address this growing need to tackle eye disease caused by oxidation damage and/or stress. The only available formulations for preventing and/or treating the results of oxidative damage and/or stress in the eye are oral formulations that deliver only small concentrations of the active ingredient to the eye after oral administration.

### SUMMARY

It has been discovered that the above-noted deficiencies can be addressed, and certain advantages attained, by the present invention. The administration of an eye drop formulation containing a high concentration of antioxidants locally to the eye would result in a higher level of protection against and/or treatment of oxidative damage to the eye. Additionally antioxidants are generally unstable and unless special precautions/controls are in place during manufacture and/or formulation these molecules rapidly revert to their inactive oxidized state when in solution. Thus, the invention described in this application addresses a critical need in the market by formulating a stable, antioxidant-containing eye drop formulation to protect eyes against oxidative damage and/or stress.

An exemplary embodiment of the invention is directed to an aqueous antioxidant-containing formulation, comprising: at least one antioxidant selected from the group consisting of L-ascorbic acid, L-glutathione, vitamin C, vitamin E, zinc, selenium and carotenoids, such as beta-carotene, lycopene, lutein, and zeaxanthin, a physiologically acceptable salt solution, and water.

In another exemplary embodiment, a concentration of the antioxidant is about 0.1 mM to about 200 mM.

In another exemplary embodiment, the aqueous antioxidant-containing formulation comprises L-ascorbic acid; and a concentration of the L-ascorbic acid can be about 0.1 mM to about 5 mM, preferably about 1.0 mM or about 2.5 mM.

In another exemplary embodiment, the aqueous antioxidant-containing formulation comprises L-glutathione; and a concentration of the L-glutathione can be about 0.1 mM to about 100 mM, preferably about 1.0 mM to about 20.0 mM.

In another exemplary embodiment, the aqueous antioxidant-containing formulation comprises L-ascorbic acid and L-glutathione.

In another exemplary embodiment, the physiologically acceptable salt solution comprises: calcium chloride, potassium chloride, magnesium sulfate, magnesium chloride, sodium chloride, sodium bicarbonate and sodium phosphate; and the physiologically acceptable salt solution can comprise: about 0.01 mM to about 25 mM calcium chloride; about 0.01 mM to about 150 mM potassium chloride; about 0.01 mM to about 20 mM monobasic potassium phosphate; about 0.01 mM to about 25 mM magnesium sulfate; about 0.01 mM to about 25 mM magnesium chloride; about 1 mM to about 175 mM sodium chloride; about 0.01 mM to about 30 mM sodium bicarbonate; and about 0.01 mM to about 25 mM dibasic sodium phosphate. Any hydration form of the salts comprised in the physiologically acceptable salt solution can be used in certain embodiments of the formulation.

In another exemplary embodiment, a pH of the aqueous antioxidant-containing formulation is about 2 to about 6, preferably about 3.5 to about 5.0.

In another exemplary embodiment, the antioxidant and the physiologically acceptable salt solution are kept separate prior to use and then mixed at point of use to create the eye drop formulation that is administered to the eyes. In other exemplary embodiments the eye drop formulation is a single solution with all components in the single formulation.

In various exemplary embodiments, the formulation can be packaged in one or more of the following formats: 1) Solution A (buffered salt solution), Solution B (L-glutathione+L-ascorbic acid), and Solutions A and B are combined at the time of use; 2) Solution A (potassium phosphate monobasic+sodium phosphate, dibasic anhydrous), Solution B (L-glutathione+L-ascorbic acid+all other salts), and Solutions A and B are combined at the time of use; 3) Solution A (potassium phosphate monobasic+sodium phosphate, dibasic anhydrous+some salts), Solution B (L-glutathione+L-ascorbic acid+other salts), and Solutions A and B are combined at the time of use; or 4) Solution A (potassium phosphate monobasic+sodium phosphate, dibasic anhydrous+salts proportionality distributed), Solution B (L-glutathione+L-ascorbic acid+salts proportionality distributed), and solutions A and B are combined at the time of use.

Another exemplary embodiment of this invention is directed to a method of preventing and/or treating ocular oxidative damage and/or stress, comprising: administering the aqueous antioxidant-containing formulation to an eye of a patient in need thereof.

Another exemplary embodiment of this invention is directed to a single- or multi-dosage form comprising the aqueous antioxidant-containing formulation. Another exemplary embodiment of this invention is directed to a kit containing the single- or multi-dosage form comprising the aqueous antioxidant-containing formulation. The kit can contain a single-dosage form comprising the aqueous antioxidant-containing formulation, and instructions for use, or a multi-dosage form comprising the aqueous antioxidant-containing formulation, and instructions for use.

Another exemplary embodiment of this invention is directed to a method for preparing an aqueous antioxidant-containing formulation, comprising: mixing at least one antioxidant selected from the group consisting of L-ascorbic acid, L-glutathione, vitamin C, vitamin E, zinc, selenium, and carotenoids, such as beta-carotene, lycopene, lutein, and zeaxanthin, and a physiologically acceptable salt solution with water, and mixing as required, to prepare a mixed formulation; and adding a pH adjusting agent to the mixed formulation, if needed, such that the mixed formulation has a pH of about 2.5 to about 5.5. For lower pH formulations (less than pH 4.5), a formulation that is not buffered or has low buffering capacity is optimal. The method can also include filtering the mixed formulation through a filter having a pore size of about 0.2 μm, and packaging and sealing the mixed formulation in sterile packaging. The method can also include sparging of the formulation with nitrogen or argon to reduce dissolved oxygen and/or filling headspace of dosage form applicator/container with argon or nitrogen.

It should be understood that the various individual aspects and features of the present invention described herein can be combined with any one or more individual aspect or feature, in any number, to form embodiments of the present invention that are specifically contemplated and encompassed by the present invention.

### DETAILED DESCRIPTION

Further aspects, features and advantages of this invention will become apparent from the detailed description which follows.

As used herein, the singular forms “a”, “an” and “the” are intended to include the plural forms as well, unless the context clearly indicates otherwise.

As used herein, “about” is a term of approximation and is intended to include minor variations in the literally stated amounts, as would be understood by those skilled in the art. Such variations include, for example, standard deviations associated with techniques commonly used to measure the amounts of the constituent elements or components of an alloy or composite material, or other properties and characteristics, up to and including a variation of ±10%. All of the values characterized by the above-described modifier “about,” are also intended to include the exact numerical values disclosed herein. Moreover, all ranges include the upper and lower limits.

Any compositions described herein are intended to encompass compositions which consist of, consist essentially of, as well as comprise, the various constituents identified herein, unless explicitly indicated to the contrary.

As used herein, the recitation of a numerical range for a variable is intended to convey that the variable can be equal to any value(s) within that range, as well as any and all sub-ranges encompassed by the broader range. Thus, the variable can be equal to any integer value or values within the numerical range, including the end-points of the range. As an example, a variable which is described as having values between 0 and 10, can be 0, 4, 2-6, 2.75, 3.19-4.47, etc.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

As used herein, the terms “patient” and “subject” are used interchangeably and include members of the animal kingdom including but not limited to human beings.

As used herein, “sterile water” includes, but is not limited to, (a) sterile water for injection, USP, (b) sterile distilled deionized water, and (c) sterile water for irrigation.

As used herein, an “antioxidant” is a substance that, when present in a mixture or structure containing an oxidizable substrate biological molecule, delays or prevents oxidation of the substrate biological molecule. For example, ascorbic acid and glutathione are antioxidants. Other suitable antioxidants include, but are not limited to antioxidant vitamins, such as provitamin A (beta-cryptoxanthin), vitamin B2 (riboflavin), vitamin B 12, vitamin C, vitamin D, vitamin E, and combinations thereof, trace mineral that exhibits antioxidant activity or that can enhance antioxidant activity of other compounds, such as copper, iron, manganese, selenium, zinc, and combinations thereof, and carotenoids such as beta-carotene, lycopene, lutein, and zeaxanthin. Any antioxidants that can be tolerated in a mammalian eye are contemplated as suitable for inclusion in the antioxidant-containing formulations of this application.

As used herein, a “balanced salt solution” is defined as an aqueous solution that is osmotically balanced to prevent acute cell or tissue damage.

As used herein, a “buffered salt solution” is defined as a balanced salt solution to which chemicals have been added to maintain a predetermined pH range and maintain ionic and isotonic balance.

As used herein, a “cellular reducing agent” is defined as a substance that loses electrons easily thereby causing other substances to be reduced chemically.

As used herein, a “buffering agent” is a chemical compound or compounds (generally a weak acid or base or combination thereof) that maintains the pH of a solution in a desired range. Examples of physiologically tolerated buffers are, for example, acetate, bicarbonate, citrate, sodium phosphate, succinate, histidine, sulfate, nitrate, and pyruvate buffers, but are not limited thereto.

As used herein, a “pH adjusting agent” is chemical compound that can raise or lower pH when added to a solution. Preferred pH adjusting agents include HCl and the like, for lowering pH, and NaHCO3, NaOH and the like, for raising pH, but are not limited thereto.

As used herein, an “isotonic solution” refers to a solution that has the same salt concentration as cells and blood.

As used herein, a “physiologically acceptable salt” refers to inorganic or organic salts, including, but not limited to, buffer salts, which are not deleterious to cell health or integrity.

As used herein, “sem” means standard error of the mean.

As described earlier, currently there are no antioxidant eye drop products to address the increasing need to tackle eye disease caused by oxidative damage and/or stress. The only available formulations for preventing and/or treating the results of oxidative damage and/or stress in the eye are oral formulations that deliver only small concentrations of the active ingredient to the eye after oral administration via systemic circulation.

Oxidative damage and/or stress is involved in the pathogenesis of many eye diseases, especially those that occur in the aging population. Examples of eye diseases arising from oxidative damage and/or stress include cataract, macular degeneration, uveitis, retinopathy, keratitis, ocular inflammation, dry eye disease, glaucoma, eye infection, and the like. Oxidative damage has been shown to: affect the optical lens; advance glycation; cause retinal capillary cell apoptosis, vascular endothelial cell dysfunction, cone degeneration, rod receptor death; and the like.

An exemplary embodiment of this application is directed to an aqueous, isotonic, and ionically balanced formulation that includes a high concentration of antioxidants. Any physiologically acceptable antioxidant known in the art can be used. In an exemplary embodiment, the aqueous antioxidant-containing formulation comprises L-ascorbic acid, L-glutathione, or L-ascorbic acid and L-glutathione. In another exemplary embodiment, L-ascorbic acid is not included in the aqueous antioxidant-containing formulation. In other exemplary embodiments, the antioxidant can include a vitamin that exhibits antioxidant activity. For example, the antioxidant can include provitamin A (beta-cryptoxanthin) vitamin B₂ (riboflavin), vitamin B12, vitamin C, vitamin D, vitamin E, and combinations thereof. The formulation can also contain a derivative antioxidant of glutathione, cysteinylglycine. In some formulations, the antioxidant can include a trace mineral that exhibits antioxidant activity or that can enhance antioxidant activity of other compounds. Examples of such trace minerals include copper, iron, manganese, selenium, zinc, and combinations thereof. In some formulations, the antioxidant can include a carotenoid that exhibits antioxidant activity. Examples of such carotenoids include beta-carotene, lycopene, lutein, and zeaxanthin. In some embodiments, the antioxidant can include a combination of two or more selected from among L-ascorbic acid, L-glutathione, provitamin A (beta-cryptoxanthin) vitamin B₂ (riboflavin), vitamin B₁₂, vitamin C, vitamin D, vitamin E, copper, iron, manganese, selenium, zinc, beta-carotene, lycopene, lutein, and zeaxanthin. In some embodiments, the antioxidant can include L-ascorbic acid and/or L-glutathione (and/or derivative cysteinylglycine) in combination with one or more of a vitamin, a trace mineral, and a carotenoid. In some embodiments, the antioxidant can include selenium when vitamin E is present as an antioxidant in the formulation. In some embodiments, the antioxidant can include L-ascorbic acid and/or L-glutathione in combination with one or more of beta-carotene, lycopene, lutein, and zeaxanthin. The antioxidant can include L-ascorbic acid and/or L-glutathione (and/or derivative cysteinylglycine) in combination with one or more of vitamin C, vitamin E, zinc, selenium and carotenoids. Any suitable carotenoid can be selected from beta-carotene, lycopene, lutein, and zeaxanthin, but is not limited thereto.

In an exemplary embodiment, the antioxidants can be selected from L-glutathione (and/or derivative cysteinylglycine), L-ascorbic acid, vitamin C, vitamin E, zinc, selenium and carotenoids and combinations thereof, but are not limited thereto. Any suitable carotenoid can be selected from beta-carotene, lycopene, lutein, and zeaxanthin, but is not limited thereto. These antioxidants prevent oxidative damage to the eyes by soaking up free radicals. The function of these antioxidants in an eye drop solution is: 1) to prevent and/or treat oxidative damage to ocular cell membranes and extracellular matrix structures; and 2) to stabilize other components of the solution by preventing oxidation, thereby improving the stability and shelf life of the product.

An exemplary embodiment of the invention is directed to an aqueous antioxidant-containing formulation, comprising: at least one antioxidant selected from the group consisting of L-ascorbic acid, L-glutathione (and/or derivative cysteinylglycine), vitamin C, vitamin E, zinc, selenium, and carotenoids, such as beta-carotene, lycopene, lutein, and zeaxanthin; and a physiologically acceptable salt solution.

In another exemplary embodiment, a concentration of the antioxidant can be about 0.1 mM to about 200 mM, about 0.2 mM to about 150 mM, about 0.3 mM to about 100 mM, about 0.4 mM to about 90 mM, about 0.5 mM to about 80 mM, about 1.0 mM to about 70 mM, about 1.5 mM to about 60 mM, about 2.0 mM to about 50 mM, about 2.5 mM to about 40 mM, about 3.0 mM to about 30 mM, about 3.5 mM to about 25 mM, about 4.0 mM to about 20 mM, about 4.5 mM to about 15 mM, about 5.0 mM to about 14 mM, about 5.5 mM to about 13 mM, about 6.0 mM to about 12 mM, about 6.5 mM to about 11 mM, about 7 mM to about 10 mM, and the like The concentration of the antioxidant is not limited hereto, and can include any values within any of the recited ranges and/or combinations thereof.

In another exemplary embodiment, the aqueous antioxidant-containing formulation comprises L-ascorbic acid; and a concentration of the L-ascorbic acid can be about 0.1 mM to about 5 mM, about 0.2 mM to about 4.5 mM, about 0.5 mM to about 4 mM, about 1 mM to about 3.5 mM, about 1.5 mM to about 3 mM, about 2 mM to about 2.5 mM, preferably about 1.0 mM or about 2.5 mM. The concentration of the L-ascorbic acid is not limited hereto, and can include any values within any of the recited ranges and/or combinations thereof.

In another exemplary embodiment, the aqueous antioxidant-containing formulation comprises L-glutathione; and a concentration of the L-glutathione can be about 0.1 mM to about 100 mM, about 0.5 mM to about 20 mM, about 1.0 mM to about 19 mM, about 1.5 mM to about 18 mM, about 2.0 mM to about 17 mM, about 2.5 mM to about 16 mM, about 3.0 mM to about 15 mM, about 3.5 mM to about 14 mM, about 4.0 mM to about 13 mM, about 4.5 mM to about 12 mM, about 5.0 mM to about 11 mM, about 5.5 mM to about 10 mM, about 6.0 mM to about 9.0 mM, about 7.0 mM to about 8.0 mM, and the like. The concentration of the L-glutathione is not limited hereto, and can include any values within any of the recited ranges and/or combinations thereof.

In another exemplary embodiment, the physiologically acceptable salt solution can comprise any combination of salts known in the art that results in a physiologically acceptable salt solution. For example, the physiologically acceptable salt solution can include a combination of salts selected from among calcium chloride, magnesium chloride, potassium chloride, sodium chloride, sodium bicarbonate, potassium phosphate, sodium phosphate, and magnesium sulfate. The amounts of the salts can vary over a large range depending on what combination of salts are used to prepare the physiologically acceptable salt solution. Exemplary concentration ranges for the salts that can be used in the physiologically acceptable salt solution can include about 0.01 mM to about 25 mM calcium chloride, about 0.01 mM to about 150 mM potassium chloride, about 0.01 mM to about 20 mM monobasic potassium phosphate, about 0.01 mM to about 25 mM magnesium sulfate, about 0.01 mM to about 25 mM magnesium chloride, about 1 mM to about 175 mM sodium chloride, about 0.01 mM to about 30 mM sodium bicarbonate, and about 0.01 mM to about 25 mM dibasic sodium phosphate. The skilled artisan can select the appropriate amount for each component from within these ranges when the combination of salts to be included in the physiologically acceptable salt solution is selected for inclusion with the antioxidant and any other components of the formulation. For example, the physiologically acceptable salt solution can include about 0.01 mM to about 10 mM calcium chloride; about 0.01 mM to about 100 mM potassium chloride; about 0.01 mM to about 10 mM monobasic potassium phosphate; about 0.01 mM to about 10 mM magnesium sulfate; about 0.01 mM to about 10 mM magnesium chloride; about 1 mM to about 155 mM sodium chloride; about 0.01 mM to about 25 mM sodium bicarbonate; and about 0.01 mM to about 10 mM dibasic sodium phosphate. If salts exist in various hydration forms, any hydration form of the salt(s) can be considered for the eye drop formulation.

The concentration of the calcium chloride can be about 0.01 mM to about 25 mM, about 0.05 mM to about 15 mM, about 0.1 mM to about 10 mM, about 0.2 mM to about 7.5 mM, about 0.3 mM to about 5 mM, about 0.4 mM to about 2.5 mM, about 0.01 to about 1.0 mM, and the like. The concentration of the potassium chloride can be about 0.01 mM to about 150 mM, about 0.05 mM to about 100 mM, about 0.1 mM to about 0.3 mM, about 75 mM, and the like. The concentration of the potassium phosphate can be about 0.01 mM to about 20 mM, about 0.02 mM to about 15 mM, about 0.03 mM to about 10 mM, about 0.04 mM to about 7.5 mM, about 0.05 mM to about 5 mM, about 0.1 mM to about 2.5 mM, about 0.2 mM to about 1 mM, and the like. The concentration of the magnesium sulfate can be about 0.01 mM to about 25 mM, about 0.02 mM to about 20 mM, about 0.03 mM to about 15 mM, about 0.04 mM to about 10 mM, about 0.05 mM to about 5 mM, about 0.1 mM to about 2.5 mM, about 0.1 mM to about 1 mM, and the like. The concentration of magnesium chloride can be about 0.01 mM to about 25 mM, about 0.02 mM to about 15 mM, about 0.03 mM to about 10 mM, about 0.04 mM to about 5 mM, about 0.05 mM to about 2.5 mM, about 0.1 mM to about 1 mM, about 0.2 mM to about 0.5 mM, and the like. The concentration of the sodium chloride can be about 0.10 mM to about 175 mM, 0.5 mM to about 150 mM, about 0.1 mM to about 125 mM, about 0.2 mM to about 120 mM, about 0.5 mM to about 100 mM, about 1 mM to about 75 mM, about 2 mM to about 50 mM, about 3 mM to about 25 mM, about 4 mM to about 15 mM, and the like. The concentration of sodium bicarbonate can be about 0.01 mM to about 30 mM, about 0.05 mM to about 25 mM, about 0.1 mM to about 15 mM, about 0.2 mM to about 10 mM, about 0.3 mM to about 5 mM, about 0.1 mM to about 1 mM, and the like. The concentration of the sodium phosphate can be about 0.01 mM to about 25 mM, about 0.02 mM to about 15 mM, about 0.03 mM to about 10 mM, about 0.04 mM to about 5 mM, about 0.05 mM to about 2.5 mM, about 0.01 mM to about 1.0 mM, and the like. The concentrations of the various components are not limited hereto, and can include any values within any of the recited ranges and/or combinations thereof.

Exemplary aqueous antioxidant-containing formulations are described herein, and other exemplary formulations can independently include hydrated or anhydrous forms of the various components, and/or can exclude one or more components.

In another exemplary embodiment, a pH of the aqueous antioxidant-containing formulation is about 2 to about 6, about 2.5 to about 5.5, about 3.0 to about 5.0, about 3.5 to about 4.5, about 3.5, about 4.0, about 5.0, and the like. The pH values are not limited hereto, and can include any values within any of the recited ranges and/or combinations thereof.

The aqueous antioxidant-containing formulations described herein can include one or more adjuvants, such as preservatives, rheology modifiers, surfactants, humectants, phospholipids, and stabilizers, as long as they do not negatively impact the antioxidant activity of the formulation. For example, a rheology modifier can be included to change the viscosity of the final formulation. Exemplary rheology modifiers include carboxymethyl cellulose, hydroxypropyl methylcellulose, hydroxyethyl cellulose, sodium hyaluronate, hyaluronic acid, a vinyl polymer compound, polyvinyl alcohol, polyvidone, poloxamer, a polyethylene glycol, a dextran, chondroitin sulfate, and alginic acid and salts thereof such as sodium alginate. Exemplary humectants include glycerin, ethylene glycol, 1,3-butylene glycol, and propylene glycol. An exemplary phospholipid is a lecithin. Other phospholipids well known to those of skill in the art as useful in ophthalmic compositions are further contemplated as useful in the present compositions. The amount of adjuvant that can be included in the formulation can be from about 0.01 wt % to about 5 wt %, based on the total weight of the formulation.

Another exemplary embodiment of this invention is directed to a method of preventing and/or treating ocular oxidative damage and/or stress, comprising: administering the aqueous antioxidant-containing formulation to an eye of a patient in need thereof.

The aqueous antioxidant-containing formulation can be dispensed into one or both eyes of a subject by any method known in the art. For example, the formulation can be dispensed into the eye as a drop that falls by gravity from a dispenser into the eye, as a spray, or by a mechanical mechanism, but is not limited thereto. Each drop can have a specified volume. For example, a drop or dosage can deliver from about 10 μL to about 250 μL or any other desired volume. In some embodiments, the dosage can be about 20 μL to about 200 μL, or about 50 μL to about 150 μL, per eye per application. In some application, installation of 1-2 drops per each eye, preferably one drop per eye, can be administered about 1 to about 7 times a day. In some embodiments, about 1 drop of the formulation can be dispensed into one or both eyes once a day, or twice a day, or about 1 to about 4 times a day. In some embodiments, about 1 drop of the formulation can be dispensed into one or both eyes in the morning and about 1 drop of the formulation can be dispensed into one or both eyes in the evening. In some embodiments, about 1 drop of the formulation can be dispensed into one or both eyes before bed. In some embodiments, the formulation can be administered throughout the day on an as needed basis to alleviate a condition that is perceived by the user.

The aqueous antioxidant-containing formulation provided herein can be administered to any subject in need thereof. In some embodiments, the subject is a mammal. The mammal can be a human, a cat, a dog, a rabbit, a bovine, an equine, an ovine, or a porcine, but is not limited thereto.

Another exemplary embodiment of this invention is directed to a single- or multi-dosage form comprising the aqueous antioxidant-containing formulation. Another exemplary embodiment of this invention is directed to a kit containing a single- or multi-dosage form comprising the aqueous antioxidant-containing formulation. The kit can include a single-dosage form comprising the aqueous antioxidant-containing formulation, and instructions for use, or a multi-dosage form comprising the aqueous antioxidant-containing formulation, and instructions for use.

Another exemplary embodiment of this invention is directed to a method for preparing an aqueous antioxidant-containing formulation, comprising: mixing at least one antioxidant selected from the group consisting of L-ascorbic acid and L-glutathione (and/or derivative cysteinylglycine) and a physiologically acceptable salt solution with water to prepare a mixed formulation; blending the formulation at a suitable speed; and adding a pH adjusting agent to the mixed formulation, if needed, such that the mixed formulation has a pH of about 2.5 to about 5.5. The method can also include filtering the mixed formulation through a filter having a pore size of about 0.2 μm, and packaging and sealing the mixed formulation in sterile packaging.

The formulation can be filtered using any suitable filter, including a filter having a pore size of about 0.2 gm, but is not limited thereto, and any suitable pore size can be used.

The formulations described in this application can be prepared using a) single run rather than multiple runs

The formulations described in this application can be packaged as a unit dose in a blow-fill-seal vial or an ophthalmic drug dispenser such as a simple squeeze bottle, but the container is not limited thereto. In some embodiments, the formulation can be delivered in an integral plastic bottle. The bottle can be designed for application by squeezing the container. Typically, the dispensing bottle can be a hand held pliable container that includes a cone-shaped nozzle, and the amount of formulation contained within the dispensing bottle that is dispensed can be adjusted by varying an inner diameter of the nozzle. Typically, instillation into the eye includes inverting the dispensing bottle and squeezing it, which causes a drop to be dispensed. The drop falls by gravity into the eye. The size of the drop can depend on the rheology and surface tension of the solution, as well as the design and diameter of the nozzle diameter. The dispenser can be designed so that the volume dispensed is independent of tilt angle of the dispensing bottle. In some embodiments, the formulation can be provided in a single-use container that is sterile until opened by the user, so that it does not need to have a preservative, and the container is disposed of after instilling the formulation into the eye. The single-use container can be provided as a single use, blow molded, plastic vial, which can include a closure or can be a twist-off vial. Any consumer-friendly packaging can be used to store and administer the formulations. The formulation can also be packaged as kits including a single dose or multiple doses. The packaging can be aseptically packaged having a Sterility Assurance Level (SAL) of about 10-3.

The formulation described in this application can be stored at temperature of up to about 32° C., with a shelf life of at least one year at room temperature.

The principles of the present invention, as well as certain exemplary features and embodiments thereof, will now be described by reference to the following non-limiting examples.

### Preparation Example

Eye drop formulations were prepared by mixing the components in the amounts listed in Table 1. Namely, appropriate amounts of the various components were weighed and then mixed with “Water for Injection” (WFI) at room temperature in a laminar flow hood to achieve the concentrations listed in Table 1. The various components were dissolved in the WFI using a magnetic stir bar and stirring at 350 rpm, or any appropriate mixing speed, until dissolution was complete. The final pH of each formulation was adjusted to pH 3.5 or pH 5.0, as listed in Table 1, using 5N HCl or 5N NaOH, respectively. The resulting solutions were then passed through a 0.21, 1 m filter while filling sterile vials with the respective formulations for use in stability studies. The same formulations shown in the example (but adjusted to pH 4 and pH 4.5) were also evaluated separately in accelerated stability studies, and these formulations, although exhibiting different stability profiles, would also have shelf-lives of at least one year. No oxygen controls were used in the preparation of these research-grade formulations, but oxygen control processes such as argon/nitrogen sparging of the formulation and/or argon/nitrogen filling of application head-space are contemplated to further increase stability of these formulations.

The formulations were then filled in borosilicate glass vials having a volume of about 13 mL. The vials were sealed with a rubber stopper followed by manual crimp capping. The formulations were stored at 40° C. in a validated stability chamber followed by periodic sampling of the stored vials for analytical testing, for example, as described in Table 2 below.

TABLE 1

| | Ex. 1A | Ex. 1B | Ex. 2B | Ex. 2B | Ex. 3A | Ex. 3B | Ex. 4A | Ex. 4B |
|---|---|---|---|---|---|---|---|---|
| Component | (mM) | (mM) | (m11) | (m11) | (mM) | (mM) | (mM) | (mM) |
| Ascorbic acid | 1 | 1 | 1 | 1 | 2.5 | 2.5 | 2.5 | 2.5 |
| Glutathione | 1 | 1 | 5 | 5 | 1 | 1 | 5 | 5 |
| Calcium chloride, | 0.14 | 0.14 | 0.14 | 0.14 | 0.14 | 0.14 | 0.14 | 0.14 |
| dihydrate | | | | | | | | |
| Potassium chloride | 0.4 | 0.4 | 0.4 | 0.4 | 0.4 | 0.4 | 0.4 | 0.4 |
| Potassium phosphate, | 0.06 | 0.06 | 0.06 | 0.06 | 0.06 | 0.06 | 0.06 | 0.06 |
| monobasic | | | | | | | | |
| Magnesium sulfate, | 0.1 | 0.1 | 0.1 | 0.1 | 0.1 | 0.1 | 0.1 | 0.1 |
| heptahydrate | | | | | | | | |
| Magnesium chloride, | 0.1 | 0.1 | 0.1 | 0.1 | 0.1 | 0.1 | 0.1 | 0.1 |
| hexahydrate | | | | | | | | |
| Sodium chloride | 8 | 8 | 8 | 8 | 8 | 8 | 8 | 8 |
| Sodium bicarbonate | 0.36 | 0.36 | 0.36 | 0.36 | 0.36 | 0.36 | 0.36 | 0.36 |
| Sodium phosphate, | 0.0271 | 0.0271 | 0.0271 | 0.0271 | 0.0271 | 0.0271 | 0.0271 | 0.0271 |
| dibasic, anhydrous | | | | | | | | |
| Water for Injection | q.s. | q.s. | q.s. | q.s. | q.s. | q.s. | q.s. | q.s. |
| _{P}H | 3.5 | 5.0 | 3.5 | 5.0 | 3.5 | 5.0 | 3.5 | 5.0 |

### Experimental Example

Sampling of the stored vials of formulations prepared as described in the Preparation Examples was carried out as follows. Two sets of vials for each formulation at pH 3.5 (Examples 1A, 2A, 3A and 4A) and pH 5.0 (Examples 1B, 2B, 3B and 4B) were tested. The vials were stored at 40° C. for 30 days. Vials were pulled from storage for testing at the times and in the numbers listed in the testing schedule of Table 2.

TABLE 2

| | | Number of Vials (stored |
|---|---|---|
| Testing Schedule | Time (±3 days) | at 40 C./75% RH |
| 1. | Time Zero | 22 |
| 2. | 3 days | 2 |
| 3. | 7 days (1 week) | 2 |
| 4. | 14 days (2 weeks) | 2 |
| 5. | 21 days (3 weeks) | 2 |
| 6. | 30 days (1 Month) | 22 |
| 7. | Reserved* | 6 |

| | |
|---|---|
| Total number of vials | 58 |

*Vials were reserved for planned analytic studies at 3, 6 and 9 months.

At each point in the testing schedule, physical properties, such as clarity, color, odor, particle count and the like, of the formulations stored in the pulled vials were examined by physical observation. Concentrations of the antioxidants were also measured, and analytical tests of the formulations in the vials were carried out according to the protocol listed in Table 3.

TABLE 3

| | | | Approx. |
|---|---|---|---|
| | | | sample |
| | Tests | Reference Test Method | size (mL) |
| 1. | Color and Odor | Physical properties, such as | n/a |
| | | clarity, color, odor, and the | |
| | | like, were examined by | |
| | | physical observation | |
| 2. | pH | USP <791> | 10 |
| 3. | Ascorbic acid | UV-VIS | 1 |
| 4. | Total Thiols | UV-VIS | |
| 5. | Particle count | USP <788> | −120 (To, |
| | | | T3o days) |
| 6. | Osmolality | USP <785> | 0.6 mL (Tzero) |

The analytical assay method for stability testing of total thiols and ascorbic acid in eyedrop formulations (UV-VIS in Table 3) are performed as follows. Ascorbic acid and glutathione (as total thiols) are assayed in eyedrop formulations using a spectrophotometric method. Both components are measured on one sample preparation. The method utilizes the wavelength programming feature of the spectrophotometer. A standard curve is generated simultaneously for each component. The slopes of each linear line are used to interpolate the concentration of each component in the sample preparation. By applying the appropriate dilution and conversion factors, the concentration of each component in the formulation can be calculated. Ascorbic acid absorbance is measured at 260 nm vs. a reagent blank. Total thiols are derivatized using a molar excess of DTNB and measuring the absorbance of the sample solution at 412 nm vs. a reagent blank.

In Experimental Example 1, all formulations exhibited similar differences in stability of ascorbic acid and glutathione when compared to each other whereas there were no substantial changes in osmolality, pH and particulates over time in any of the formulations.

In some of the evaluated formulations, ascorbic acid appeared to be more stable at the lower pH of 3.5 compared to pH 5. For example: ascorbic acid appears to be more stable at the lower pH of Example 1A compared to the higher pH of Example 1B, and at the lower pH of Example 3A compared to the higher pH of Example 3B. In other formulations, the stability of ascorbic acid was maintained regardless of pH (Examples 2A, 2B, 4A and 4B). This was an unexpected property of the formulations of this application compared to the known stability trends for ascorbic acid. Another unexpected result of the formulations of this application was the destabilizing effect of higher molar ratios of ascorbic acid on glutathione.

It is expected that all formulations with the exception of the formulations of Examples 1B and 3B will have a shelf-life of at least 1 year. The formulations of this application were prepared without oxygen controls, but it is contemplated that the formulations can also be prepared under conditions that control exposure to oxygen thereby conferring an even longer shelf-life. For formulations prepared using oxygen controlled processes, formulations of this application, including Examples 1B and 3B, would be expected to have a shelf-life of at least 1 year.

Preclinical tests for predicting human eye tolerability are also contemplated. One such testing protocol is the Draize test. The Draize test is a standard acute ocular toxicity test, which provides a method for assessing the irritation potential of materials that come in contact with human eyes, including eye care products and drugs designed specifically for topical ophthalmic use. The current Draize method involves the instillation of about 10 μL of a test liquid (or about 10 mg of a test solid) into the lower conjunctival cul-de-sac, followed by a saline rinse. Observations of various criteria (i.e., corneal opacity and area of corneal involvement, conjunctival hyperemia, chemosis, ocular discharges, and iris abnormalities) are taken at predefined intervals, for example, at about 1 hour, about 24 hours, about 48 hours, about 72 hours, about 7 days, and about 21 days after administration. A slit-lamp examination can be added to allow better assessment of corneal lesions, and topical fluorescein can be applied to reveal any cornel ulceration. Optical or ultrasonic pachymetry can also be implemented to measure the extent of corneal thickening.

The Draize test is usually performed in rabbits due to their large eyes, well-described anatomy, ease of handling, relatively low cost, and ready availability. Inventors of this application do not expect to observe any adverse effects in any of the test subjects after exposure to the aqueous antioxidant formulations described in this application. Inventors of this application also expect the antioxidant aqueous formulation of this application to be safe and tolerable when administered to a subject in need thereof, including but not limited to a mammalian subject.

As various changes could be made in the above methods and compositions without departing from the scope of the invention, it is intended that all matter contained in the above description shall be interpreted as illustrative and not in a limiting sense. Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C. § 112, paragraph 6, unless the term “means” is explicitly used.

1-37. (canceled)

38. An aqueous antioxidant-containing formulation, comprising: at least one antioxidant selected from the group consisting of L-ascorbic acid, L-glutathione, cysteinylglycine, vitamin C, vitamin E, zinc, selenium, beta-carotene, lycopene, lutein, and zeaxanthin; and a physiologically acceptable salt solution.

39. The aqueous antioxidant-containing formulation of claim 38, wherein a pH of the aqueous antioxidant-containing formulation is about 2 to about 6.

40. The aqueous antioxidant-containing formulation of claim 39, wherein the pH of the aqueous antioxidant-containing formulation is about 2.5 to less than 4.5.

41. The aqueous antioxidant-containing formulation of claim 38, wherein a concentration of the antioxidant is about 0.1 mM to about 200 mM, and the aqueous antioxidant-containing formulation comprises L-ascorbic acid, whereby a concentration of the L-ascorbic acid is about 0.1 mM to about 5 mM.

42. The aqueous antioxidant-containing formulation of claim 38, wherein the aqueous antioxidant-containing formulation comprises L-glutathione and a concentration of the L-glutathione is about 0.1 mM to about 15 mM.

43. The aqueous antioxidant-containing formulation of claim 38, wherein the aqueous antioxidant-containing formulation comprises L-ascorbic acid and L-glutathione, whereby a concentration of the L-ascorbic acid is about 0.1 mM to about 5 mM and a concentration of the L-glutathione is about 0.1 mM to about 15 mM.

44. The aqueous antioxidant-containing formulation of claim 38, wherein the physiologically acceptable salt solution comprises a combination of two or more selected from among calcium chloride, potassium chloride, magnesium sulfate, magnesium chloride, sodium chloride, sodium bicarbonate and sodium phosphate.

45. The aqueous antioxidant-containing formulation of claim 44, wherein the physiologically acceptable salt solution comprises:
i. about 0.01 mM to about 25 mM calcium chloride, dihydrate;
ii. about 0.01 mM to about 150 mM potassium chloride;
iii. about 0.01 mM to about 20 mM monobasic potassium phosphate;
iv. about 0.01 mM to about 25 mM magnesium sulfate, heptahydrate;
v. about 0.01 mM to about 25 mM magnesium chloride, hexahydrate;
vi. about 1 mM to about 175 mM sodium chloride;
vii. about 0.01 mM to about 30 mM sodium bicarbonate;
viii. about 0.01 mM to about 25 mM dibasic sodium phosphate; and
ix. water.

46. The aqueous antioxidant-containing formulation of claim 38, wherein said antioxidant and said physiologically acceptable salt solution are separate prior to use.

47. A method for preparing an aqueous antioxidant-containing formulation, comprising:
mixing at least one antioxidant selected from the group consisting of L-ascorbic acid, L-glutathione, vitamin C, vitamin E, zinc, selenium, beta-carotene, lycopene, lutein, and zeaxanthin and a physiologically acceptable salt solution with water to prepare a mixed formulation;
stirring the mixed formulation; and
adding a pH adjusting agent to the mixed formulation such that the mixed formulation has a pH of about 2.5 to about 5.5.

48. The method of claim 47, wherein a pH of the aqueous antioxidant-containing formulation is about 3.5 to about 5.0.

49. The method of claim 47, wherein the pH of the aqueous antioxidant-containing formulation is about 2.5 to less than 4.5.

50. The method of claim 47, further comprising filtering the mixed formulation through a filter having a pore size of about 0.2 μm.

51. The method of claim 47, wherein a concentration of the antioxidant is about 0.1 mM to about 200 mM.

52. The method of claim 47, wherein the aqueous antioxidant-containing formulation comprises L-ascorbic acid and a concentration of the L-ascorbic acid is about 0.1 mM to about 5 mM.

53. The method of claim 47, wherein the aqueous antioxidant-containing formulation comprises L-glutathione and a concentration of the L-glutathione is about 0.1 mM to about 15 mM.

54. The method of claim 53, wherein the concentration of the L-ascorbic acid is about 1.0 mM or about 2.5 mM.

55. The method of claim 47, wherein the aqueous antioxidant-containing formulation comprises L-ascorbic acid and L-glutathione, and a concentration of the L-ascorbic acid is about 0.1 mM to about 5 mM and a concentration of the L-glutathione is about 0.1 mM to about 15 mM.

56. The method of claim 47, wherein the physiologically acceptable salt solution comprises: calcium chloride, potassium chloride, magnesium sulfate, magnesium chloride, sodium chloride, sodium bicarbonate and sodium phosphate.

57. The method of claim 56, wherein the physiologically acceptable salt solution comprises:
i. about 0.01 mM to about 25 mM calcium chloride, dihydrate;
ii. about 0.01 mM to about 150 mM potassium chloride;
iii. about 0.01 mM to about 20 mM monobasic potassium phosphate;
iv. about 0.01 mM to about 25 mM magnesium sulfate, heptahydrate;
v. about 0.01 mM to about 25 mM magnesium chloride, hexahydrate;
vi. about 1 mM to about 175 mM sodium chloride;
vii. about 0.01 mM to about 30 mM sodium bicarbonate;
viii. about 0.01 mM to about 25 mM dibasic sodium phosphate; and
ix. water.
